(19) United States
(12) Patent Application Publication
(10) Pub. No.: US 2023/0126905 A1
(43) Pub. Date: Apr. 27, 2023

(54) LEAD LOCKING SYSTEM AND METHOD FOR IMPLANTABLE STIMULATORS

(71) Applicant: INCUBE LABS, LLC, San Jose, CA (US)

(72) Inventors: Elmar FISCHER, Boerne, TX (US); Charles Gregory NELSON, New Braunfels, TX (US); Paul SPEHR, San Antonio, TX (US); Kenneth R. COSGROVE, San Jose, CA (US)

(73) Assignee: INCUBE LABS, LLC, San Jose, CA (US)

(21) Appl. No.: 17/915,280

(22) PCT Filed: Jan. 27, 2021
(86) PCT No.: PCT/US2021/015276

§ 371 Date: Sep. 28, 2022

Related U.S. Application Data
(60) Provisional application No. 63/002,183, filed on Mar. 30, 2020.

(51) Int. Cl. A61N 1/375

(52) U.S. Cl. CPC A61N 1/3752

(57) ABSTRACT

A header lock assembly includes a header lock pin and mating lead lock collar that provide a releasable coupling for attaching one or more leads to a device. The header lock assembly is configured to engage the lead to assure closure while not applying undue pressure on the lead. One or more notches in the header lock pin allow the lead to pass the header lock pin unimpeded while the header lock assembly is in an unlocked configuration. Rotation of the header lock pin engages a circumferential groove in the lead lock collar to secure the lead to the header. A slot in the top of the header lock pin provides an interface surface with a lead locking tool for rotating the header lock pin. The slot orientation provides a visual indication of the header lock assembly being in a locked or unlocked configuration.

### CROSS-REFERENCE TO RELATED APPLICATIONS

This application claims priority to, and the benefit of, U.S. Provisional Patent Application No. 63/002,183 filed Mar. 30, 2020, which is incorporated herein by reference in its entirety for all purposes.

### STATEMENT REGARDING FEDERALLY SPONSORED RESEARCH OR DEVELOPMENT

This invention was made with Government support under W81XWH-15-C-0066, awarded by U.S. Department of Defense, U.S. Army Medical Research Acquisition Activity. The Government has certain rights in the invention.

### BACKGROUND

### Technical Field

Embodiments of the present description relate to systems and methods for electrical stimulation and/or therapy. More specifically, embodiments of the present description relate to coupling mechanism for releasably locking electrical leads to an implantable controller.

### Background Discussion

A system may include an electrical device and leads detachably electrically connected to the device. For example, a device may provide medical treatment via electrical signals delivered through one or more leads (e.g., neurostimulation). The device and/or the leads may be implanted.

A lead may be inserted into an opening in a header assembly of a device for electrical connection to the device, and can be held in position by one or more set screws. Such set screws are generally inserted through small holes in the header assembly and tightened to engage an outer surface of the lead.

Such existing systems can lead to several problems. The set screw is prone to be overtightened, which may result in deformation of the lead and/or electrical shorting. Also, there is significant opportunity for leaks through the set screw holes. Additionally, the surgical crew must secure each of multiple leads one at a time, increasing overall surgical time.

Thus, there is a need for a mechanism for coupling leads to devices that can provide greater reliability, less labor during implantation, elimination of potential leak paths, and enhanced overall performance.

### SUMMARY

An aspect of the technology of the present description is a system including a header lock assembly with a header lock pin and mating lead lock collar that provide a secure releasable coupling for attaching leads to a controller. The header lock assembly engages the lead to assure closure while not applying undue pressure on the lead. One or more notches in the header lock pin allow the lead to pass by unimpeded while in the open position. Rotating the header lock pin engages a circumferential groove in the lead lock collar to secure the lead. Correspondingly, rotating the header lock pin more, or in the opposite direction, disengages the circumferential groove in the lead lock collar to release the lead from the header. An engagement surface in the form of a slot in the top of the header lock pin provides a surface for purchase/interface with a lead locking tool for turning the header lock pin. The orientation of the slot provides a visual indication as to whether the releasable coupling is in a locked or unlocked configuration. The header and header lock pin may be structured such that rotation of the header lock pin can secure multiple leads concurrently (e.g., simultaneously). Fluid ingress paths are minimized or eliminated, so that no septum plug is needed.

Further aspects of the technology described herein will be brought out in the following portions of the specification, wherein the detailed description is for the purpose of fully disclosing embodiments of the technology without placing limitations thereon.

### BRIEF DESCRIPTION OF THE DRAWINGS

The technology described herein will be more fully understood by reference to the following drawings which are for illustrative purposes only:

FIG. 1A shows a perspective view of an embodiment of a device including a quarter-turn releasable coupling assembly for releasably attaching a lead to the device, with the releasable coupling assembly in an unlocked configuration and the lead removed.

FIG. 1B shows a perspective view of an embodiment of the device of FIG. 1A, with the releasable coupling assembly in a locked configuration and the lead engaged.

FIG. 2 shows a side view of an embodiment of a header lock pin such as may be used in the releasable coupling assembly of FIG. 1A and FIG. 1B.

FIG. 3 shows a perspective view of an embodiment of the releasable coupling assembly in isolation.

FIG. 4A shows a side view of the releasable coupling assembly of FIG. 3 according to an embodiment, viewed down a long axis of the lead, with the releasable coupling assembly in an unlocked configuration.

FIG. 4B shows a side view of the releasable coupling assembly of FIG. 3 according to an embodiment, viewed down a long axis of the lead, with the releasable coupling assembly in a locked configuration.

FIG. 5 shows a side view of an embodiment of the device of FIG. 1A with the lead removed.

FIG. 6A shows a cross-sectional plan view through section 6-6 of FIG. 5, where section 6-6 is taken in a horizontal plane through an uppermost receiving aperture of a header housing while the releasable coupling assembly is in an unlocked configuration, according to an embodiment.

FIG. 6A shows a cross-sectional plan view through section 6-6 of FIG. 5, where section 6-6 is taken in a horizontal plane through an uppermost receiving aperture of a header housing while the releasable coupling assembly is in a locked configuration, according to an embodiment.

FIG. 7A shows a partial cutout plan view through section 6-6 (FIG. 5) of FIG. 5, with the lead assembly inserted in a header assembly and the releasable coupling assembly is in an unlocked configuration, according to an embodiment.

FIG. 7B shows a partial cutout plan view through section 6-6 (FIG. 5) of FIG. 5, with the lead assembly inserted in a header assembly and the releasable coupling assembly is in an unlocked configuration, according to an embodiment.

### DETAILED DESCRIPTION

Embodiments of the present technology are directed to a device incorporating a quarter-turn header lock assembly having a header lock pin and mating lead lock collar that provides a secure, releasable coupling for attaching leads to the device. The systems and methods of the present technology are particularly suited for interfacing leads to a biomedical device for delivering treatment to a target tissue region within a body. An example of such a biomedical device is a neurostimulator, which may be implantable, where the leads include one or more electrical conductors which may be electrically connected at a distal end of the lead to one or more electrodes for stimulating the target tissue region. However, it is appreciated that the systems and methods of the present technology may be implemented for any system incorporating releasable coupling of leads.

The introduction of a biomedical device of the present technology may be performed by a doctor or medical professional, who may use an imaging modality (e.g., fluoroscopy, ultrasound, or endoscopic image capture) or sensing devices that allow attending personnel to position the treatment section of the device at the target location. The implantation process may involve use of a separate implantation device (e.g., an introducer) which can provide a conduit or path for the lead device as it is introduced. Once the lead device is positioned at the target location, the implantation device can be removed. It may then be desirable for the lead device, or a portion thereof, to retain its position for days, weeks, months, or years.

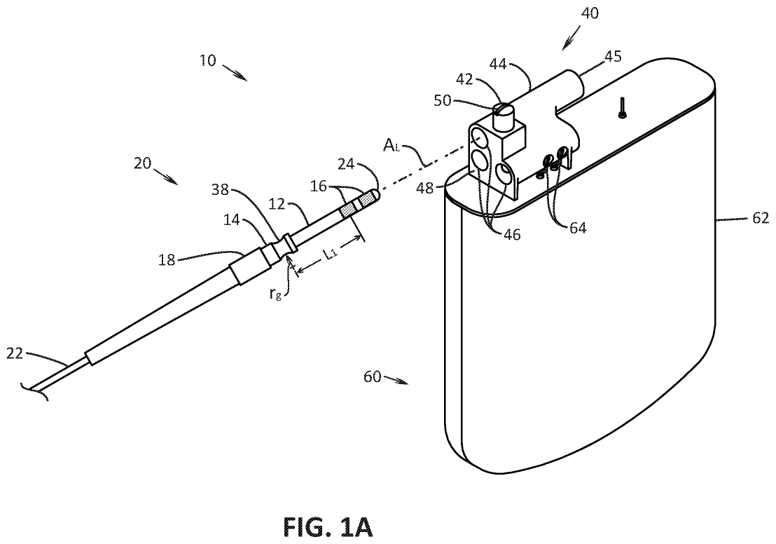

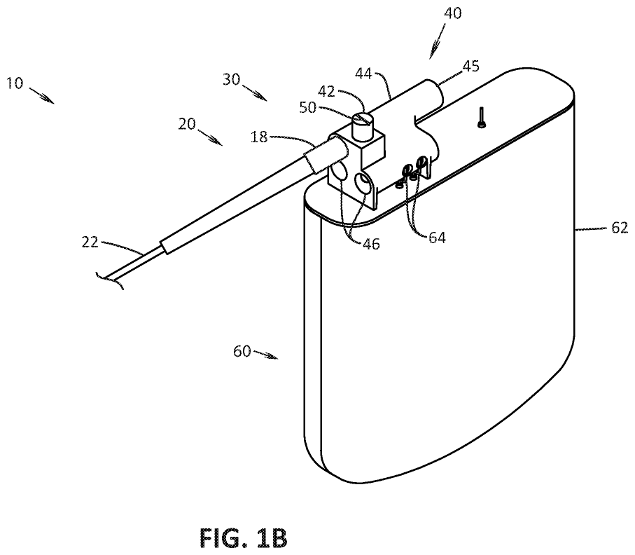

FIG. 1A through FIG. 7B illustrate embodiments of a medical device in the form of an implantable stimulator 10 including a lead assembly 20 and a controller 60. A header assembly 40 is attached to a controller housing 62 of the controller 60.

In an embodiment, lead assembly 20 is a lead for providing therapeutic electrical stimulation or other treatment energy signal to a target tissue region within or on a body.

In FIG. 1A, implantable stimulator 10 is shown in a first configuration with lead assembly 20 separated from header assembly 40. A header housing 44 of header assembly 40 includes one or more receiving apertures 46 (here, three are shown) for receiving lead assembly 20. Particularly, lead assembly 20 is assembled with controller 60 via insertion of a proximal lead body 12 of lead assembly 20 into one of receiving apertures 46. While three receiving apertures 46 are shown in the embodiment illustrated in FIG. 1A and FIG. 1B, header housing 44 may incorporate any number of receiving apertures 46 to accommodate the number of lead assemblies 20 needed for the treatment to be applied. Each receiving aperture 46 is a cavity that is closed at an end 45 and open at a face 48 of header housing 44. Apertures 46 are shaped to accommodate a shape of the lead assembly 20 to be received; here, proximal lead body 12 is approximately cylindrical with a hemisphere-shaped tip, and therefore a cavity of at least one of apertures 46 will have an approximately cylindrical shape and potentially a hemisphere-shaped end at closed end 45 of header housing 44. The lead assembly 20 and a corresponding aperture 46 can have other shapes, such as oval or polygonal in cross-section.

More than one header housing 44 may be disposed at various locations on controller housing 62, such as to expand a number of lead assemblies 20 that may be coupled to controller 60, to achieve a desired overall shape or dimensions of assembled implantable stimulator 10, or to allow for coupling of lead assemblies 20 from different angles.

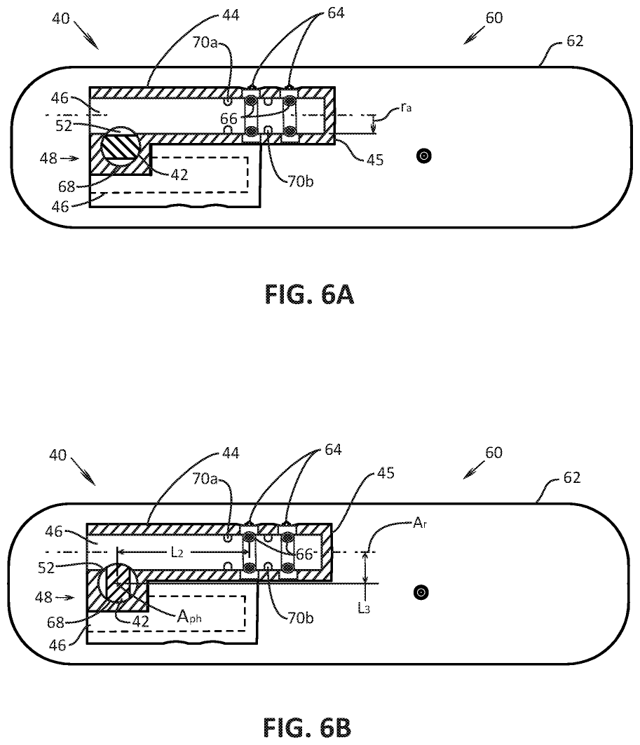

Header lock pin 42 is disposed in a pin hole 68 (see FIG. 6A and FIG. 6B) running into a top portion of header housing 44 orthogonal to receiving apertures 46. As discussed in further detail below with respect to FIG. 6A and FIG. 6B, header lock pin 42 extends through pin hole 68 and into each of receiving apertures 46 when oriented in a locking position.

Header assembly 40 includes a releasable coupling assembly 30 (see FIG. 1B through FIG. 4B) having a header lock pin 42 and mating lead lock collar 14 for releasably attaching a lead assembly 20 to controller 60 (e.g., IPG).

In the embodiment illustrated, proximal lead body 12 has a body portion (here, shown with an approximately cylindrical shape) including a proximal tip 24 and a pair of spaced-apart annular or semi-annular lead electrical contacts 16 disposed just distal to proximal tip 24. Lead lock collar 14, described in more detail below, is disposed at the distal extent of proximal lead body 12.

The distal end of lead lock collar 14 is coupled to a lead line 22 via a lead body 18, which in some embodiments has a larger diameter than lead lock collar 14. Lead line 22 can be a thin, elongate, flexible line that extends from controller 60 to one or more treatment electrodes (not shown) at a distal end (also not shown) of lead line 22 for location at a target treatment site remotely located from controller 60. It is appreciated that treatment electrodes may take any number of different forms, including ring, planar, spherical, or other shapes or configurations.

FIG. 1B shows a perspective view of implantable stimulator 10 with lead assembly 20 installed in one of the upper receiving apertures 46 of header assembly 40 with releasable coupling assembly 30 in a locked configuration after an angular turn of header lock pin 42. In an embodiment, header lock pin 42 includes one or more engagement features to allow header lock pin 42 to more easily be rotated. In the embodiment illustrated in FIG. 1B (and other figures), an engagement feature is provided in the form of a single slot 50 for receiving a rotational tool (e.g., disc or screwdriver). One or more other engagement features may be used additionally or alternatively. In the embodiment illustrated, locking is accomplished by way of a quarter-turn of header lock pin 42, as indicated by the approximately 90-degree angle orientation difference of slot 50 between FIG. 1A and FIG. 1B. Other embodiments use locking angles greater than or less than 90 degrees.

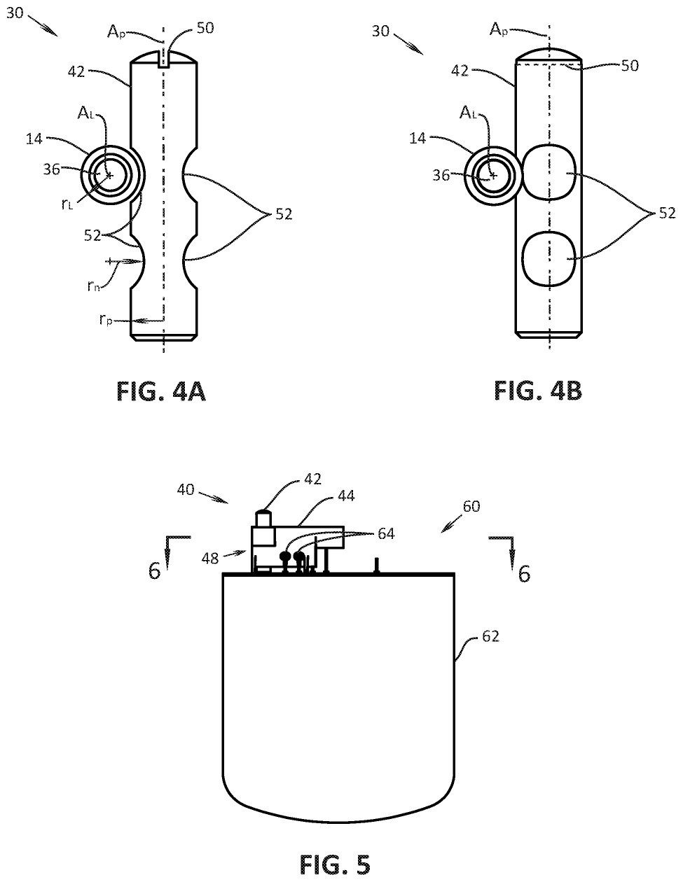

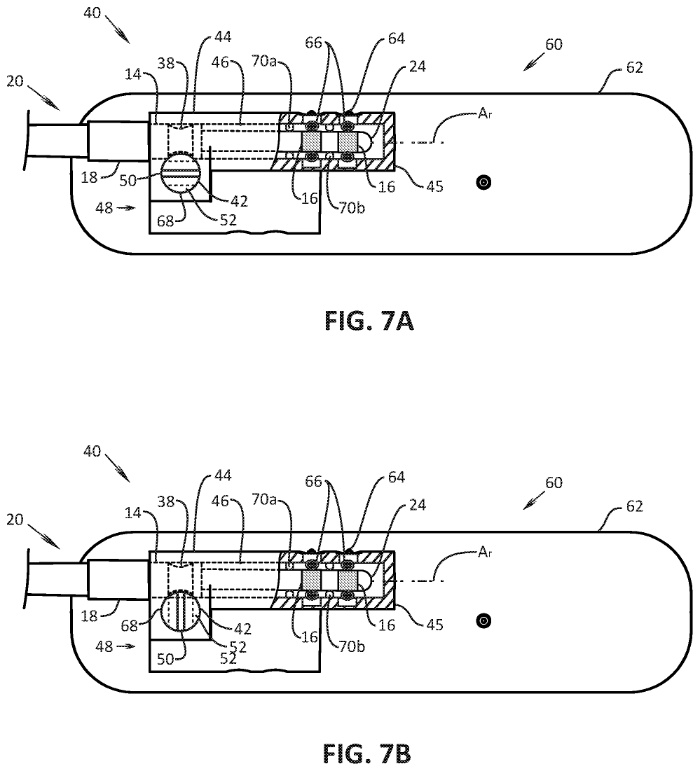

An outer dimension of lead lock collar 14 (FIG. 1A, FIG. 3 through FIG. 4B, FIG. 7A and FIG. 7B) may be sized to closely match, or be slightly smaller than, a corresponding inner dimension of receiving aperture 46 to seal off the cavity of receiving aperture 46 from the external environment, thus preventing infusion of fluids or other unwanted substances into the cavity. In an embodiment, proximal lead body 12 (FIG. 1A) is inserted into receiving aperture 46 to a depth such that lead body 18 abuts face 48 of header housing 44, and thus lead body 18 may also provide a seal against face 48 of header assembly 40. In some embodiments, an O-ring or similar gasket (not shown) may be positioned on lead lock collar 14 just proximal to lead body 18, or on lead body 18 just proximal to lead lock collar 14, to provide further sealing of receiving aperture 46 against face 48 of header assembly 40 upon installation of lead assembly 20 into receiving aperture 46.

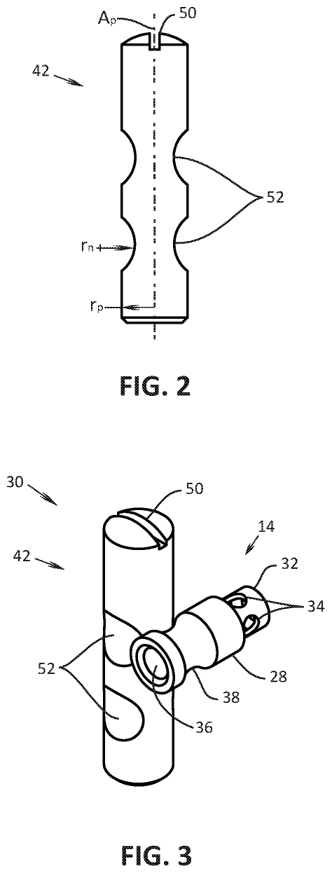

FIG. 2 shows a side view of an embodiment of header lock pin 42 suitable for use in releasable coupling assembly 30 (FIG. 1B, FIG. 3, FIG. 4A and FIG. 4B). Header lock pin 42 includes a cylindrical body having an outer surface defined by radius rₚ, and transverse notches 52 in the outer surface. In the embodiment shown in FIG. 2, notches 52 are arcuate in shape from a side view and have a radius rₙ with an axis that is transverse to, or orthogonal to, a long axis Aₚ of header lock pin 42. In the embodiment shown in FIG. 2 through FIG. 4B, notches 52 are positioned as two pairs of opposing notches spaced apart along a length of header lock pin 42. While this configuration allows for simultaneous lock (or release) of up to four leads within header housing 44, it is appreciated that any number of notches 52 may be included to accommodate various numbers of lead assemblies 20, and various orientations of notches 52 may be implemented to accommodate various configurations of header housing 44 (FIG. 1A, FIG. 1B, and FIG. 5 through FIG. 7B).

In some embodiments, a top surface of header lock pin 42 includes slot 50 to allow for manipulation of header lock pin 42 and thus orientation of notches 52 by rotation of header lock pin 42 along axis Aₚ. In the embodiment shown in FIG. 1A through FIG. 4B, slot 50 is in the form of a linear slot through a portion of the top of header lock pin 42. This allows for purchase with an adjustment tool (e.g., flat head screwdriver). While slot 50 may take other shapes to accommodate other types of tools (e.g., Allen wrench or Phillips head screwdriver), the use of a linear slot 50 as shown in shown in FIG. 1A through FIG. 4B also provides a visual indication of the orientation of header lock pin 42, and thus an indication of whether releasable coupling assembly 30 is in a locked or unlocked configuration.

FIG. 3 through FIG. 4B illustrate various views of releasable coupling assembly 30. In the perspective view of FIG. 3, header lock pin 42 and lead lock collar 14 are shown in isolation in a locked position to illustrate engagement surfaces of releasable coupling assembly 30. FIG. 4A shows a side view of lead assembly 20 (FIG. 1A, FIG. 1B, FIG. 7A and FIG. 7B) along axis A_{L} of releasable coupling assembly 30, with header lock pin 42 in an unlocked position such that lead lock collar 14 is disengaged or released from header housing 44. FIG. 4B shows the same side view as FIG. 4A, with header lock pin 42 in a locked position such that lead lock collar 14 is engaged and thus secured to header housing 44.

As shown in FIG. 3, lead lock collar 14 is a tubular structure having a central channel 36 along lead axis A_{L} and a shaft 32 proximal to a main portion 28 of lead lock collar 14. Central channel 36 defines an aperture suitable to allow passage of electrical conductors (not shown) for electrical coupling to lead electrical contacts 16 (FIG. 1A, FIG. 7A and FIG. 7B) on proximal lead body 12 (FIG. 1A). Such electrical conductors may be disposed within a sheath (not shown) and may pass through the sheath to couple to lead electrical contacts 16.

In an embodiment, shaft 32 has a cylindrical surface having a smaller radius than an outer surface of main portion 28, and shaft 32 defines radially-oriented thru-holes 34. Thru-holes 34 can provide for improved adhesion to lead body 18 and/or improved securing of electrical conductors within central channel 36, by, for example, disposing a material (e.g., plastic or other polymeric material) into thru-holes 34 before assembly with lead body 18 (FIG. 1A, FIG. 1B, FIG. 7A and FIG. 7B). In other embodiments (not shown), shaft 32 and central channel 36 may be larger such that the shaft fits over a lead wire casing (not shown).

In an embodiment, central channel 36 has an inner diameter larger than an outer diameter of proximal lead body 12 (FIG. 1A) such that proximal lead body 12 may be assembled into lead lock collar 14 at an end distal to proximal tip 24 (FIG. 1A, FIG. 7A and FIG. 7B) of proximal lead body 12.

Lead lock collar 14 defines a circumferential groove 38 circumscribing lead lock collar 14. In an embodiment, circumferential groove 38 is in the form of an arcuate surface with radius r_{g} (FIG. 1A). In some embodiments, the center of radius r_{g} is located at a specified axial distance L₁ from a first one of lead electrical contacts 16 (FIG. 1A, FIG. 7A and FIG. 7B) along the longitudinal axis A_{L} of lead assembly 20. The distance L₁ also corresponds to a location of a first mating header electrical contact 66 in header housing 44 (see FIGS. 6A through 7B).

Referring to FIG. 4A, to allow insertion of lead assembly 20 (FIG. 1A, FIG. 1B, FIG. 7A and FIG. 7B) into header housing 44 (FIG. 1A and FIG. 1B), or to release lead assembly 20 from header housing 44, slot 50 of header lock pin 42 is rotated about header lock pin axis Aₚ a quarter turn (90°) so that notches 52 are aligned with lead axis A_{L}. In an embodiment, radius rₙ of notches 52 is equal to or greater than a radius r_{L} of main portion 28 (FIG. 3) of lead lock collar 14, and notches 52 are located (when header lock pin 42 is installed in header housing 44) such that the center of radius rₙ is coincident or closely aligned with lead axis A_{L} when lead assembly 20 is installed in receiving aperture 46. In this manner, notches 52 allow lead lock collar 14 to freely traverse receiving aperture 46 past header lock pin 42 until proximal lead body 12 (FIG. 1A) of lead assembly 20 is at an appropriate depth into receiving aperture 46, and lead body 18 is at or near face 48 (FIG. 1A, FIG. 5 through FIG. 7B) of header housing 44 (see FIG. 1B). Lead lock collar 14 may be composed of a hard, biocompatible material (e.g., one or more metals such as stainless steel or titanium, one or more alloys, one or more polymers, or combinations or assemblies thereof).

FIG. 3 and FIG. 4B show releasable coupling assembly 30 in a locked configuration. Header lock pin 42 is rotated about header lock pin axis Aₚ to an orientation such that notches 52 are a quarter turn (90°) away from circumferential groove 38 (FIG. 1A, FIG. 3, FIG. 7A and FIG. 7B) of lead lock collar 14. This orients outer radius rₚ of header lock pin 42 into circumferential groove 38 to form the locked configuration. Slot 50 of header lock pin 42 is transverse to lead axis A_{L} (FIG. 4B), providing visual indication of the orientation of header lock pin 42 and locked configuration of releasable coupling assembly 30.

As seen in FIG. 1A, FIG. 1B, FIG. 5, and FIG. 6A through FIG. 7B, header assembly 40 provides an interface for one or more lead assemblies 20 to be releasably coupled to controller 60 via header housing 44, which may be attached to or integral with controller housing 62. In an embodiment, header housing 44 includes a cast epoxy header defining receiving apertures 46. It is appreciated that header housing 44 may include any number of materials (e.g., biocompatible polymeric materials having insulative properties), and may be fabricated via various production techniques (e.g., machining or injection molding).

An electrical feedthrough wire 64 extends from inside controller housing 62 into header housing 44 to header electrical contacts 66 to provide electrical coupling between installed lead assemblies 20 and various components of controller 60. In an embodiment, feedthrough wire 64 is 80/20 platinum-iridium wire with polymeric coating.

FIG. 5 through FIG. 7B show additional views illustrating header assembly 40. FIG. 5 shows a side view of implantable stimulator 10 (with lead assemblies 20 omitted). FIG. 6A and FIG. 6B show cross-sectional plan views of implantable stimulator 10 (with lead assemblies 20 omitted) through section 6-6 of FIG. 5, where section 6-6 is taken in a horizontal plane through an uppermost one of receiving apertures 46 of header housing 44. FIG. 6A shows header lock pin 42 in an unlocked configuration, while FIG. 6B shows header lock pin 42 in a locked configuration. FIG. 7A and FIG. 7B show partial cutout plan views through header housing 44 with lead assembly 20 installed and with header lock pin 42 in an unlocked and locked configuration, respectively.

Header electrical contacts 66 are disposed at spaced-apart locations within receiving apertures 46 to engage lead electrical contacts 16 when lead assembly 20 is installed (FIG. 7A and FIG. 7B). There may be more or fewer header electrical contacts 66 than lead electrical contacts 16, as a lead assembly 20 may be used with different header assemblies 40 and/or different controllers 60, and a header assembly 40 may be used with different lead assemblies 20, such as to manufacture a single controller 60 design for use in two or more therapeutic applications. In an embodiment in which there is a mismatched number of header electrical contacts 66 and lead electrical contacts 16, at least one lead electrical contact 16 will align with a corresponding header electrical contact 66 when lead assembly 20 is locked in header assembly 40. In an embodiment, header electrical contacts 66 are compliant helical ring contacts (e.g. Bal-Seal from Bal Seal Engineering, Inc.); however, a number of shapes or configurations may be employed to provide a conductive coupling with lead electrical contacts 16 of lead assembly 20.

Pin hole 68 opens from an upper surface of header housing 44 and has an axis Aₚₕ (FIG. 6B) that is orthogonal to receiving apertures 46 (i.e., axis Aₚₕ runs vertically with respect to axis Aᵣ of horizontally disposed receiving apertures 46 in header housing 44). In the embodiment shown in FIG. 6A through FIG. 7B (and FIG. 1A), pin hole 68 is disposed between receiving apertures 46. Pin hole 68 may be sized to have a slight interference fit with header lock pin 42 when installed, such that header lock pin 42 remains retained in pin hole 68 and maintains its rotational orientation within pin hole 68. A rotational stop (not shown) may also be included between header lock pin 42 and header housing 44 so that rotation of header lock pin 42 is restricted to a limited rotation (e.g., quarter-turn) between locked and unlocked orientations.

In an embodiment, the location of pin hole 68 (and correspondingly header lock pin 42 when installed within header housing 44) is positioned to properly locate lead assembly 20 within aperture 46 and allow for ingress/egress of lead assembly 20 in the unlocked configuration of FIG. 6A, as well as corresponding locking of lead assembly 20 in the locked configuration of FIG. 6B. As shown in FIG. 6A, a portion of pin hole 68 extends into each receiving aperture 46. When header lock pin 42 is oriented with notches 52 in line with aperture axis Aᵣ as in FIG. 6A and FIG. 7A, header lock pin 42 does not protrude into aperture 46. This allows for lead lock collar 14 to pass freely into and out of receiving aperture 46. When header lock pin 42 is oriented with notches 52 transverse with aperture axis Aᵣ as in FIG. 6B, the outer surface (with radius rₚ) of header lock pin 42 now partially extends into receiving aperture 46 and engages with circumferential groove 38, preventing significant movement of lead lock collar 14 within aperture 46 and thus locking lead lock collar 14 in its axial location along aperture axis Aᵣ.

A distance L₂ (FIG. 6B) along aperture axis Aᵣ between pin hole 68 axis Aₚₕ and header electrical contacts 66 may be sized to correspond closely with the distance L₁ between the centerline of circumferential groove 38 radius r_{g} and lead electrical contacts 16 of lead assembly 20 (FIG. 1A). Thus, when header lock pin 42 is rotated into the locked position shown in FIG. 6B and FIG. 7B, it will have the effect of aligning header electrical contacts 66 with lead electrical contacts 16 to form an electrical coupling, even if lead assembly 20 was not exactly aligned in the aperture prior to rotation of header lock pin 42. A distance L₃ between pin hole 68 axis Aₚₕ and aperture axis Aᵣ may be sized to press the outer surface of header lock pin 42 into circumferential groove 38 when in the locked position shown in FIG. 6B and FIG. 7B, which would result in pressing lead electrical contacts 16 onto header electrical contacts 66. In an embodiment, header electrical contacts 66 are deformable to spring load upon such compression to maintain electrical contact with lead electrical contacts 16 even in the event of motion, vibration, or other force that could otherwise disturb such contact. One or more O-rings may also be included within receiving aperture 46 to prevent or limit migration of fluid or debris toward header electrical contacts 66. For example, a first O-ring 70a may be disposed at a location proximal to header electrical contacts 66 to prevent fluid migration from the opening of receiving aperture 46 and/or pin hole 68 toward header electrical contacts 66. A second O-ring 70b may be disposed at a location between header electrical contacts 66 to prevent fluid migration from one header electrical contact 66 to another. Receiving aperture 46 is shown in FIG. 6A through FIG. 7B in a simplistic manner for clarity, and many internal features of receiving aperture 46 (e.g., polymeric spacers) may also be included that are not detailed in the figures.

In an embodiment, an O-ring, gasket, or similar compressible sealing element (not shown) is disposed between face 48 of header housing 44 and lead body 18 (when lead assembly 20 is installed). The rotation of header lock pin 42 into the locked position may serve to pull proximal lead body 12 (FIG. 1A) into receiving aperture 46, resulting in compression of the sealing element between lead body 18 and abutting face 48. This acts to seal off the cavity of receiving aperture 46 from the external environment, thus preventing infusion of fluids or other unwanted substances into the cavity.

The position of header lock pin 42 and notches 52 in header housing 44, as shown in FIG. 1A through FIG. 7B, allows for locking of all lead assemblies 20 (e.g., three receiving apertures 46 in the configuration of FIG. 1A) to be locked or unlocked concurrently with a one quarter-turn rotation of header lock pin 42.

Header housing 44 may be configured to house multiple header lock pins 42 to accommodate coupling with additional lead assemblies 20, or to engage multiple header lock pins 42 with multiple circumferential grooves of lead lock collar 14.

As seen in FIG. 7A, when header lock pin 42 is oriented with notches 52 in line with aperture axis Aᵣ (see also FIG. 6A), visual indication/verification of such orientation and unlocked configuration is provided by slot 50, which is also shown in line with aperture axis Aᵣ. Correspondingly depicted in FIG. 7B, when header lock pin 42 is oriented with notches 52 orthogonal with aperture axis Aᵣ (see also FIG. 6B), visual indication/verification of such orientation and locked configuration is provided by slot 50, which is also shown orthogonal with aperture axis Aᵣ.

As can be seen from the figures and the description above, header assembly 40 provides a consistent and repeatable coupling to positively engage lead assembly 20 to assure closure while not applying undue pressure on lead assembly 20. The one or more notches 52 in header lock pin 42 allow lead assembly 20 to pass by unimpeded while header lock pin 42 is oriented in the unlocked position. Turning header lock pin 42 by 90 degrees (i.e., a quarter turn) engages circumferential groove 38 in lead lock collar 14 to securely lock lead assembly 20 with visual confirmation provided by slot 50. Correspondingly, turning the header lock pin 42 by 90 degrees (either in the same direction or in the opposite direction) disengages header lock pin 42 from circumferential groove 38 in lead lock collar 14 to release lead assembly 20 from header housing 44. Also, header housing 44 and header lock pin 42 are structured such that a single turn of header lock pin 42 can secure multiple lead assemblies 20 concurrently, electrically engaging lead assemblies 20 with controller 60 while minimizing or eliminating potential fluid ingress paths.

Although described with respect to medical applications, the locking features described herein are suitable for a variety of devices, such as for underwater devices or equipment sensitive to dust or humidity or air movement (e.g., audio equipment connections).

From the description herein, it will be appreciated that the present disclosure encompasses multiple embodiments which include, but are not limited to, the following:

1. An apparatus, comprising: a current source; a housing coupled to the current source having one or more receiving apertures; a first lead having a proximal end and a distal end comprising one or more electrodes for applying an signal to a target site; the proximal end of the first lead comprising one or more electrical contacts for coupling to the current source; the proximal end of the first lead further comprising a lead lock collar disposed in proximity to the one or more electrical contacts; the lead lock collar comprising a cylindrical surface comprising a groove extending at least partially around a circumference of the cylindrical surface; a lock pin disposed in a pin aperture of the housing, the pin aperture extending into a portion of at least one of the one or more receiving apertures; the lock pin comprising a pin axis and an outer circumferential surface having one or more notches disposed transverse to the pin axis; wherein the lock pin is configured to rotate within the pin aperture about the pin axis between a first angular orientation and a second angular orientation; wherein a first transverse notch of the one or more transverse notches aligns with a first receiving aperture within the housing such that the outer circumferential surface of the lock pin extends into the receiving aperture in the first angular orientation, and the first receiving aperture is substantially free of the outer circumferential surface of lock pin as a result of alignment of the first transverse notch with the first receiving aperture in the second angular orientation; wherein a proximal end of the first lead is configured to be inserted into the first receiving aperture to electrically couple the first lead to the current source; wherein in the first angular orientation, the outer circumferential surface of the lock pin couples with the groove of the lead lock collar to restrict axial motion of and thereby retain the first lead within the first receiving aperture; and wherein in the second angular orientation, the alignment of the first transverse notch of the lock pin with the first receiving aperture frees axial motion of the first lead within the first receiving aperture to allow ingress and egress of the first lead within the first receiving aperture.

2. The apparatus, system, or method of any of the preceding or subsequent embodiments, wherein the lock pin further comprises a second transverse notch on the outer circumferential surface of the lock pin; wherein the housing further comprises a second receiving aperture adjacent the first receiving aperture for receiving a second lead; and wherein the first and second transverse notches are aligned with respective receiving apertures of the housing such that both first and second receiving apertures are substantially free of the lock pin when positioned in the second angular orientation, and lock the first and second leads in respective first and second receiving apertures in the first angular orientation.

3. The apparatus, system, or method of any of the preceding or subsequent embodiments, wherein a single 90° rotation of the lock pin from the second angular orientation to the first angular orientation simultaneously locks corresponding first and second leads when positioned in respective first and second receiving apertures.

4. The apparatus, system, or method of any of the preceding or subsequent embodiments, wherein the second transverse notch is located on an opposing side of the outer circumferential surface of the lock pin from the first transverse notch; and wherein the pin hole is located in between the first and second receiving apertures to align the first and second transverse notches with first and second receiving apertures when the pin is positioned in the second angular orientation.

5. The apparatus, system, or method of any of the preceding or subsequent embodiments, wherein the lock pin comprises a slot disposed on a surface of the lock pin that is accessible externally from the housing; wherein the slot provides an engagement surface to allow coupling with a tool to provide for rotation of the pin between the first orientation and the second orientation; and wherein the slot provides visual identification of the orientation of the lock pin between the first angular orientation and the second angular orientation.

6. The apparatus, system, or method of any of the preceding or subsequent embodiments, wherein the groove in the lead lock collar comprises a circumferential groove entirely circumscribing the circumferential surface of the lead lock collar; and wherein the lock pin is configured to engage the lead lock collar independently of the angular orientation of the first lead along a longitudinal axis of the first lead while positioned in the in the first receiving aperture.

7. The apparatus, system, or method of any of the preceding or subsequent embodiments, wherein the circumferential groove comprises an arcuate shape having a groove radius, the groove radius substantially matching a radius of the outer circumferential surface of the lock pin.

8. The apparatus, system, or method of any of the preceding or subsequent embodiments, wherein the one or more transverse notches comprise an arcuate shape having a notch radius, the notch radius substantially matching a radius of the one or more receiving apertures.

9. The apparatus, system, or method of any of the preceding or subsequent embodiments, wherein the pin hole and circumferential groove are located such that when the lock pin is rotated to the first orientation, the proximal end of the first lead is axially aligned in the first receiving aperture to align with and contact the one or more electrical contacts of the first lead with one or more inner electrical contacts disposed in the housing, the one or more inner electrical contacts being coupled to the current source.

10. A system for releasably locking one or more leads to a controller, comprising: a housing operably coupled to a controller configured for delivery of electrical current to at least one lead, the lead comprising a distal end having one or more electrodes for applying an signal to a target site, and a proximal end having one or more electrical contacts for coupling to the controller; a lead lock collar coupled to the least one lead, the lead lock collar being disposed in proximity to the one or more electrical contacts and comprising a cylindrical surface comprising a groove extending at least partially around a circumference of the cylindrical surface; wherein the housing comprises one or more receiving apertures for receiving one or more respective leads; wherein the housing further comprises a lock pin disposed in a pin aperture of the housing, the pin aperture extending into a portion of at least one of the one or more receiving apertures, the lock pin comprising a pin axis and an outer circumferential surface having one or more transverse notches disposed transverse to the pin axis, the lock pin being configured to rotate about the pin axis within the pin aperture between a first angular orientation and a second angular orientation; wherein a first transverse notch of the one or more transverse notches aligns with a first receiving aperture within the housing such that the outer circumferential surface of the lock pin extends into the receiving aperture in the first angular orientation and the first receiving aperture is substantially free of the outer circumferential surface of lock pin as a result of alignment of the first transverse notch with the first receiving aperture in the second angular orientation; wherein in the first orientation, the outer circumferential surface of the lock pin couples with the groove of the lead lock collar to restrict axial motion of and thereby retain a first lead within the first receiving aperture; and wherein in the second orientation, the alignment of the first transverse notch of the lock pin with the first receiving aperture frees axial motion of the first lead within the first receiving aperture to allow ingress and egress of the first lead within the first receiving aperture.

11. The apparatus, system, or method of any of the preceding or subsequent embodiments: wherein the lock pin further comprises a second transverse notch on the outer circumferential surface of the lock pin; wherein the housing further comprises a second receiving aperture adjacent the first receiving aperture for receiving a second lead; and wherein the first and second transverse notches are aligned with respective receiving apertures of the housing such that both first and second receiving apertures are substantially free of the lock pin when positioned in the second angular orientation and such that the first and second leads are locked in respective first and second receiving apertures in the first angular orientation.

12. The apparatus, system, or method of any of the preceding or subsequent embodiments, wherein a single 90° rotation of the lock pin from the second angular orientation to the first angular orientation simultaneously locks corresponding first and second leads when positioned in respective first and second receiving apertures.

13. The apparatus, system, or method of any of the preceding or subsequent embodiments, wherein the second transverse notch is located on an opposing side of the outer circumferential surface of the lock pin from the first transverse notch; and wherein the pin hole is located in between the first and second receiving apertures to align the first and second transverse notches with first and second receiving apertures when the pin is positioned in the second angular orientation.

14. The apparatus, system, or method of any of the preceding or subsequent embodiments, wherein the lock pin comprises a slot disposed on a surface of the lock pin that is accessible externally from the housing; wherein the slot provides an engagement surface to allow coupling with a tool to provide for rotation of the pin between the first orientation and the second orientation; and wherein the slot provides visual identification of the orientation of the lock pin between the first angular orientation and the second angular orientation.

15. The apparatus, system, or method of any of the preceding or subsequent embodiments, wherein the groove in the lead lock collar comprises a circumferential groove entirely circumscribing the circumferential surface of the lead lock collar; and wherein the lock pin is configured to engage the lead lock collar independently of the angular orientation along a longitudinal axis of the first lead while positioned in the in the first receiving aperture.

16. The apparatus, system, or method of any of the preceding or subsequent embodiments: wherein the circumferential groove comprises an arcuate shape having a groove radius, the groove radius substantially matching a radius of the outer circumferential surface of the lock pin; and wherein the pin hole and circumferential groove are located such that when the lock pin is rotated to the first angular orientation, the proximal end of the lead is axially aligned in the first receiving aperture to align with and contact the one or more electrical contacts of the first lead with one or more inner electrical contacts disposed in the housing, the one or more inner electrical contacts being coupled to the controller.

17. The apparatus, system, or method of any of the preceding or subsequent embodiments, wherein the one or more transverse notches comprise an arcuate shape having a notch radius, the notch radius substantially matching a radius of the one or more receiving apertures.

18. A method for releasably locking one or more leads to a controller, comprising: providing a housing operably coupled to a controller configured for delivery of electrical current to at least one lead, the lead comprising a distal end having one or more electrodes for applying an signal to a target site, and a proximal end having one or more electrical contacts for coupling to the controller; providing a lead lock collar coupled to the least one lead, the lead lock collar being disposed in proximity to the one or more electrical contacts and comprising a cylindrical surface comprising a groove extending at least partially around a circumference of the cylindrical surface; inserting a first lead in a first receiving aperture of the one or more respective receiving apertures; wherein the housing further comprises a lock pin disposed in a pin aperture of the housing, the pin aperture extending into a portion of at least one of the one or more receiving apertures, the lock pin comprising a pin axis and an outer circumferential surface having one or more transverse notches disposed transverse to the pin axis, the lock pin being rotatable about the pin axis between a first angular orientation and a second angular orientation; wherein the first receiving aperture is substantially free of the outer circumferential surface of the lock pin as a result of alignment of the first transverse notch with the first receiving aperture while the first lead is being inserted with the lock pin in the second angular orientation; and rotating the lock pin about the pin axis within the pin aperture from the second angular orientation to the first angular orientation to align a first transverse notch of the one or more transverse notches with the first receiving aperture within the housing such that the outer circumferential surface of the lock pin extends into the first receiving aperture in the first angular orientation to couple the outer circumferential surface of the lock pin with the groove of the lead lock collar to restrict axial motion of and thereby retain the first lead within the first receiving aperture.

19. The apparatus, system, or method of any of the preceding or subsequent embodiments, wherein the lock pin further comprises a second transverse notch on the outer circumferential surface of the lock pin and the housing further comprises a second receiving aperture adjacent the first receiving aperture, the method comprising: while the lock pin is positioned in the second angular orientation, inserting a second lead in the second receiving aperture; wherein the first and second transverse notches are aligned with respective first and second receiving apertures of the housing such that both first and second receiving apertures are substantially free of the lock pin when positioned in the second angular orientation; and wherein a single rotation of the lock pin from the second angular orientation to the first angular orientation simultaneously locks corresponding first and second leads when positioned in respective first and second receiving apertures.

20. The apparatus, system, or method of any of the preceding or subsequent embodiments, wherein the circumferential groove comprises an arcuate shape having a groove radius, the groove radius substantially matching a radius of the outer circumferential surface of the lock pin; and wherein the pin hole and circumferential groove are located such that when the lock pin is rotated to the first orientation, the proximal end of the first lead is axially positioned in the receiving aperture to align with and contact the one or more electrical contacts of the first lead with one or more inner electrical contacts disposed in the housing, the one or more inner electrical contacts being coupled to the controller.

As used herein, the singular terms “a,” “an,” and “the” may include plural referents unless the context clearly dictates otherwise. Reference to an object in the singular is not intended to mean “one and only one” unless explicitly so stated, but rather “one or more.”

As used herein, the terms “substantially” and “about” are used to describe and account for small variations. When used in conjunction with an event or circumstance, the terms can refer to instances in which the event or circumstance occurs precisely as well as instances in which the event or circumstance occurs to a close approximation. When used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. For example, “substantially” aligned can refer to a range of angular variation of less than or equal to ±10°, such as less than or equal to ±5°, less than or equal to 4°, less than or equal to 3°, less than or equal to ±2°, less than or equal to 1°, less than or equal to ±0.5°, less than or equal to 0.1°, or less than or equal to ±0.05°.

The foregoing description of various embodiments of the technology of the present disclosure has been presented for purposes of illustration and description. It is not intended to limit the technology of the present disclosure to the precise forms disclosed. Many modifications, variations and refinements are possible. For example, embodiments of the device can be sized and otherwise adapted for releasable coupling of any electrical lead to a corresponding electrical component such as a controller, amplifier, processor, or other electronic circuitry. Further, numerous equivalents to the specific devices and methods described herein are possible. Such equivalents are considered to be within the scope of the technology of the present disclosure and are covered by the appended claims below.

Elements, characteristics, or acts from one embodiment can be readily recombined or substituted with one or more elements, characteristics or acts from other embodiments to form numerous additional embodiments within the scope of the technology of the present disclosure. Moreover, elements that are shown or described as being combined with other elements, can, in various embodiments, exist as standalone elements. Further embodiments of the disclosure also contemplate the exclusion or negative recitation of an element, feature, characteristic, value, step or wherever said element, feature, agent, characteristic, value, step or the like is positively recited. Hence, the scope of the technology of the present disclosure is not limited to the specifics of the described embodiments, but is instead limited solely by the appended claims.

Although the description herein contains many details, these should not be construed as limiting the scope of the disclosure but as merely providing illustrations of some of the presently preferred embodiments. Therefore, it will be appreciated that the scope of the disclosure fully encompasses other related embodiments.

All structural and functional equivalents to the elements of the disclosed embodiments that are known to those of ordinary skill in the art are expressly incorporated herein by reference and are intended to be encompassed by the present claims. Furthermore, no element, component, or method step in the present disclosure is intended to be dedicated to the public regardless of whether the element, component, or method step is explicitly recited in the claims. No claim element herein is to be construed as a “means plus function” element unless the element is expressly recited using the phrase “means for”. No claim element herein is to be construed as a “step plus function” element unless the element is expressly recited using the phrase “step for.”

What is claimed is:

1. An apparatus, comprising:
a current source;
a housing coupled to the current source having one or more receiving apertures;
a first lead having a proximal end and a distal end;
the distal end of the first lead comprising one or more electrodes for applying an signal to a target site;
the proximal end of the first lead comprising one or more electrical contacts for coupling to the current source;
the proximal end of the first lead further comprising a lead lock collar disposed in proximity to the one or more electrical contacts;
the lead lock collar comprising a cylindrical surface comprising a groove extending at least partially around a circumference of the cylindrical surface;
a lock pin disposed in a pin aperture of the housing, the pin aperture extending into a portion of at least one of the one or more receiving apertures;
the lock pin comprising a pin axis and an outer circumferential surface having one or more transverse notches disposed transverse to the pin axis;
wherein the lock pin is configured to rotate within the pin aperture about the pin axis between a first angular orientation and a second angular orientation;
wherein a first transverse notch of the one or more transverse notches aligns with a first receiving aperture within the housing such that the outer circumferential surface of the lock pin extends into the receiving aperture in the first angular orientation, and the first receiving aperture is substantially free of the outer circumferential surface of the lock pin as a result of an alignment of the first transverse notch with the first receiving aperture in the second angular orientation;
wherein a proximal end of the first lead is configured to be inserted into the first receiving aperture to electrically couple the first lead to the current source;
wherein in the first angular orientation, the outer circumferential surface of the lock pin couples with the groove of the lead lock collar to restrict axial motion of and thereby retain the first lead within the first receiving aperture; and
wherein in the second angular orientation, the alignment of the first transverse notch of the lock pin with the first receiving aperture frees axial motion of the first lead within the first receiving aperture to allow ingress and egress of the first lead within the first receiving aperture.

2. The apparatus of claim 1:
wherein the lock pin further comprises a second transverse notch on the outer circumferential surface of the lock pin;
wherein the housing further comprises a second receiving aperture adjacent the first receiving aperture for receiving a second lead; and
wherein the first and second transverse notches are aligned with respective receiving apertures of the housing such that both first and second receiving apertures are substantially free of the lock pin when positioned in the second angular orientation and the first and second leads are locked in respective first and second receiving apertures in the first angular orientation.

3. The apparatus of claim 2, wherein a single 90° rotation of the lock pin from the second angular orientation to the first angular orientation simultaneously locks corresponding first and second leads when positioned in respective first and second receiving apertures.

4. The apparatus of claim 2, wherein the second transverse notch is located on an opposing side of the outer circumferential surface of the lock pin from the first transverse notch; and
wherein the pin aperture is located in between the first and second receiving apertures to align the first and second transverse notches with first and second receiving apertures when the lock pin is positioned in the second angular orientation.

5. The apparatus of claim 1, wherein the lock pin comprises a slot disposed on a surface of the lock pin that is accessible externally from the housing;
wherein the slot provides an engagement surface to allow coupling with a tool to provide for rotation of the lock pin between the first orientation and the second orientation; and
wherein the slot provides visual identification of the orientation of the lock pin between the first angular orientation and the second angular orientation.

6. The apparatus of claim 1, wherein the groove in the lead lock collar comprises a circumferential groove entirely circumscribing a circumferential surface of the lead lock collar; and
wherein the lock pin is configured to engage the lead lock collar independently of the angular orientation of the first lead along a longitudinal axis of the first lead while positioned in the in the first receiving aperture.

7. The apparatus of claim 6, wherein the circumferential groove comprises an arcuate shape having a groove radius, the groove radius substantially matching a radius of the outer circumferential surface of the lock pin.

8. The apparatus of claim 7, wherein the pin aperture and circumferential groove are located such that when the lock pin is rotated to the first orientation, the proximal end of the first lead is axially positioned in the first receiving aperture to align with and contact the one or more electrical contacts of the first lead with one or more inner electrical contacts disposed in the housing, the one or more inner electrical contacts being coupled to the current source.

9. The apparatus of claim 1, wherein the one or more transverse notches comprise an arcuate shape having a notch radius, the notch radius substantially matching a radius of the one or more receiving apertures.

10. A system for releasably locking one or more leads to a controller, comprising:
a housing operably coupled to a controller configured for delivery of electrical current to at least one lead, the lead comprising a distal end having one or more electrodes for applying a signal to a target site, and a proximal end having one or more electrical contacts for coupling to the controller;
a lead lock collar coupled to the least one lead, the lead lock collar being disposed in proximity to the one or more electrical contacts and comprising a cylindrical surface comprising a groove extending at least partially around a circumference of the cylindrical surface;
wherein the housing comprises one or more receiving apertures for receiving one or more respective leads;
wherein the housing further comprises a lock pin disposed in a pin aperture of the housing, the pin aperture extending into a portion of at least one of the one or more receiving apertures, the lock pin comprising a pin axis and an outer circumferential surface having one or more transverse notches disposed transverse to the pin axis, the lock pin being configured to rotate about the pin axis within the pin aperture between a first angular orientation and a second angular orientation;
wherein a first transverse notch of the one or more transverse notches aligns with a first receiving aperture within the housing such that the outer circumferential surface of the lock pin extends into the receiving aperture in the first angular orientation and the first receiving aperture is substantially free of the outer circumferential surface of the lock pin as a result of an alignment of the first transverse notch with the first receiving aperture in the second angular orientation;
wherein in the first orientation, the outer circumferential surface of the lock pin couples with the groove of the lead lock collar to restrict axial motion of and thereby retain a first lead within the first receiving aperture; and
wherein in the second orientation, the alignment of the first transverse notch of the lock pin with the first receiving aperture frees axial motion of the first lead within the first receiving aperture to allow ingress and egress of the first lead within the first receiving aperture.

11. The system of claim 10:
wherein the lock pin further comprises a second transverse notch on the outer circumferential surface of the lock pin;
wherein the housing further comprises a second receiving aperture adjacent the first receiving aperture for receiving a second lead; and
wherein the first and second transverse notches are aligned with respective receiving apertures of the housing such that both first and second receiving apertures are substantially free of the lock pin when positioned in the second angular orientation, and lock the first and second leads in respective first and second receiving apertures in the first angular orientation.

12. The system of claim 11, wherein a single 90° rotation of the lock pin from the second angular orientation to the first angular orientation simultaneously locks corresponding first and second leads when positioned in respective first and second receiving apertures.

13. The system of claim 11, wherein the second transverse notch is located on an opposing side of the outer circumferential surface of the lock pin from the first transverse notch; and
wherein the pin aperture is located in between the first and second receiving apertures to align the first and second transverse notches with first and second receiving apertures when the lock pin is positioned in the second angular orientation.

14. The system of claim 10, wherein the lock pin comprises a slot disposed on a surface of the lock pin that is accessible externally from the housing;
wherein the slot provides an engagement surface to allow coupling with a tool to provide for rotation of the lock pin between the first orientation and the second orientation; and
wherein the slot provides visual identification of the orientation of the lock pin between the first angular orientation and the second angular orientation.

15. The system of claim 10, wherein the groove in the lead lock collar comprises a circumferential groove entirely circumscribing the circumferential surface of the lead lock collar; and
wherein the lock pin is configured to engage the lead lock collar independently of the angular orientation along a longitudinal axis of the first lead while positioned in the in the first receiving aperture.

16. The system of claim 15:
wherein the circumferential groove comprises an arcuate shape having a groove radius, the groove radius substantially matching a radius of the outer circumferential surface of the lock pin; and
wherein the pin aperture and circumferential groove are located such that when the lock pin is rotated to the first angular orientation, the proximal end of the lead is axially aligned in the first receiving aperture to align with and contact the one or more electrical contacts of the first lead with one or more inner electrical contacts disposed in the housing, the one or more inner electrical contacts being coupled to the controller.

17. The system of claim 10, wherein the one or more transverse notches comprise an arcuate shape having a notch radius, the notch radius substantially matching a radius of the one or more receiving apertures.

18. A method for releasably locking one or more leads to a controller, comprising:
providing a housing operably coupled to a controller configured for delivery of electrical current to at least one lead, the lead comprising a distal end having one or more electrodes for applying an signal to a target site, and a proximal end having one or more electrical contacts for coupling to the controller;
providing a lead lock collar coupled to the least one lead, the lead lock collar being disposed in proximity to the one or more electrical contacts and comprising a cylindrical surface comprising a groove extending at least partially around a circumference of the cylindrical surface;
inserting a first lead in a first receiving aperture of the one or more respective receiving apertures;
wherein the housing further comprises a lock pin disposed in a pin aperture of the housing, the pin aperture extending into a portion of at least one of one or more receiving apertures, the lock pin comprising a pin axis and an outer circumferential surface having one or more transverse notches disposed transverse to the pin axis, the lock pin being rotatable about the pin axis between a first angular orientation and a second angular orientation;
wherein the first receiving aperture is substantially free of the outer circumferential surface of the lock pin as a result of alignment of the first transverse notch with the first receiving aperture while the first lead is being inserted with the lock pin in the second angular orientation; and
rotating the lock pin about the pin axis within the pin aperture from the second angular orientation to the first angular orientation to align a first transverse notch of the one or more transverse notches with the first receiving aperture within the housing such that the outer circumferential surface of the lock pin extends into the first receiving aperture in the first angular orientation to couple the outer circumferential surface of the lock pin with the groove of the lead lock collar to restrict axial motion of and thereby retain the first lead within the first receiving aperture.

19. The method of claim 18, wherein the lock pin further comprises a second transverse notch on the outer circumferential surface of the lock pin and the housing further comprises a second receiving aperture adjacent the first receiving aperture, the method comprising:
while the lock pin is positioned in the second angular orientation, inserting a second lead in the second receiving aperture;
wherein the first and second transverse notches are aligned with respective first and second receiving apertures of the housing such that both first and second receiving apertures are substantially free of the lock pin when positioned in the second angular orientation; and
wherein a single rotation of the lock pin from the second angular orientation to the first angular orientation simultaneously locks corresponding first and second leads when positioned in respective first and second receiving apertures.

20. The method of claim 18, wherein the groove comprises an arcuate shape having a groove radius, the groove radius substantially matching a radius of the outer circumferential surface of the lock pin; and
wherein the pin aperture and groove are located such that when the lock pin is rotated to the first orientation, the proximal end of the first lead is axially aligned in the receiving aperture to align with and contact the one or more electrical contacts of the first lead with one or more inner electrical contacts disposed in the housing, the one or more inner electrical contacts being coupled to the controller.
